(19) United States
(12) Patent Application Publication
(10) Pub. No.: US 2006/0266917 A1
(43) Pub. Date: Nov. 30, 2006

(54) Wireless Power Transmission System

(76) Inventors: Sisinio F. Baldis, San Diego, CA (US); Hector A. Baldis, Pleasanton, CA (US); Jessica J. Baldis, San Diego, CA (US)

Correspondence Address: MANUEL F. DE LA CERRA, 6885 CATAMARAN DRIVE, CARLSBAD, CA, 92011, US

(21) Appl. No.: 11/419,453

(22) Filed: May 19, 2006

Related U.S. Application Data
(60) Provisional application No. 60/684,203, filed on May 23, 2005.

(51) Int. Cl. C12Q 1/68; H01J 40/00

(52) U.S. Cl. 250/200

(57) ABSTRACT

A novel method for wireless power transmission that comprises a transmitter and a receiver is disclosed. The receiver does not require an independent power source and is comprised of an optical feedback to the transmitter, and therefore does not require a separate communication channel to the transmitter. The transmitter uses the optical feedback to locate and track the receiver. The transmitter can optionally employ a macro adjusters and micro adjusters that direct the beam onto the receiver for optimal power transmission. The system also optionally has a tight loop beam detector to enhance safety of the system. Either the receiver and/or the transmitter may also encode data on the energy transmission, resulting in one-way or two-way data transmission.

### 1 CLAIM OF PRIORITY

The present patent application claims priority to U.S. provisional patent application 60/684,203 filed on May 23, 2005 by inventors Sisinio F. Baldis, Hector A. Baldis, and Jessica J. Baldis. The content of the provisional patent application is incorporated herein by reference.

### 2 FIELD OF THE INVENTION

The present invention relates to the wireless transmission of power.

### 3 BACKGROUND OF THE INVENTION

Wireless power transmission is generally used in situations where providing a physical wire to the intended target is difficult or even impossible. For example, moving objects present a particularly difficult problem in transmitting energy. In the past, moving objects like trains, used heavy infrastructure (including overhead power cables) to supply the needed energy. Therefore, there exists a need for efficient and safe wireless power transmission that avoids costly infrastructure.

Recently, there has been much experimentation with microwave wireless power. Because microwaves are very long waves, the detection, reception and transmission equipment must also be very large. This limits its application and drives up costs. Also, microwave energy transmission can travel through objects, which is both a benefit and detriment. It is beneficial because the energy transmission need not be in line of sight; however, it can travel through objects and cause damage to unintended (and unseen) targets.

Magnetic induction is another wireless power transmission, which is used heavily in radio frequency identification (RFID). A magnetic field is generated and the receiver, using a coil, transforms the magnetic energy into electrical energy. The main drawback here, however, is that the magnetic field is generally emitted radially, meaning that the amount of power received drops off as a function of the square of the inverse of the distance from the transmitter (i.e., 1/r²). So magnetic induction power transmission is only possible over very short distances and much of the energy transmitted is wasted.

To overcome these problems, others have used a light source (such as a laser) to focus energy onto a power receiver. The benefit of a focused light beam is that power can be efficiently transmitted over much larger distances. Having a focused light power beam introduces a new set of problems, however: (1) the power beam must be precisely focused on a target over very long distances and (2) because the power beam is high in energy, it must be maintained on the target, lest it diverges and causes unintended damage.

U.S. Pat. No. 4,078,747 discloses using high power lasers to wirelessly transmit power to supply electric trains, and to transmit solar energy generated from satellites in space. While using a light source has the benefit of reduced energy losses and the ability to direct the light source, care must be taken when the environment may have obstructions that may be damaged by a high-intensity energy beam. People may be especially susceptible to a high intensity power beam, and U.S. Pat. No. 4,08,747 does not present an effective method to safely use the high intensity beam.

U.S. Application No. 2004/0227057 A1 addresses some of these problems by using a separate wireless communications channel to locate and manage the exchange of power between the transmitter and the receiver and to detect when a break in the beam has occurred. The shortcomings of the '057 application are that the receiver requires an independent source of power and an entirely new channel of communication. So if the receiver loses its power, then energy transmission is impossible. Also, if the new channel of communication fails, either through equipment malfunction or interference, then effective energy transmission is again impossible. Finally, the '057 application does not address how to accurately find and position the beam on the receiver, and how to maintain that position on the receiver.

Therefore, a need exists for a high-energy wireless transmission system wherein the receiver does not require an independent source of power or an independent channel of communication, but can nevertheless assist the transmitter in locating the receiver passively. A need further exists for a wireless energy transmission system that can accurately find and position an energy beam on the receiver and maintain the beam's position for optimal power transmission.

### 4 SUMMARY OF THE INVENTION

The present invention provides an improved method for wireless power transmission that comprises a transmitter and a passive receiver, wherein the receiver does not require an independent power source and the receiver provides optical feedback to the transmitter.

An embodiment of the transmitter described herein uses a low power electromagnetic beam to locate the receiver. While locating the receiver, the transmitter scans an area for the receiver using the low power beam. To overcome the issues described above regarding accurately locating the transmitter, the transmitter employs adaptive optical focusing and multistage scanning. In an embodiment, an optical focusing component will be used in conjunction with the multistage beam scanner allowing for micro adjustment of the beam placement. A coarse beam will initially scan the area until the receiver is located. Because the initial beam is coarse, the area can be rapidly scanned. Location is confirmed by passive optical feedback from the receiver. After the initial location is made, the transmitter focuses the beam, and through optics, can micro-adjust the beam position for additional accuracy and placement on the target area of the receiver. Once the receiver is located, optical focusing will be used to focus the beam for maximum power transmission, and the beam source may be switched into high power transmission. Optionally, if at any time the transmitter's beam detector ceases to detect the reflected beam (i.e., the optical feedback) or if the intensity distribution seen at the position sensor is not as expected, it shuts down the high power beam. This tightly coupled feedback loop enhances safety and is an improvement over the prior art which employs a separate channel for feedback and beam shutdown.

In one example, the receiver described herein comprises a beam partitioner and a retro-reflector, such that a portion of the beam received by the receiver is reflected back to the transmitter. In an embodiment, the receiver comprises a mask or other variable reflectance such that the reflected beam characteristics will differ depending on the location of the beam on the receiver.

By analyzing these characteristics, the transmitter can micro-adjust the beam position until the desired reflected characteristics are attained, which would correspond to the optimal position of the beam on the receiver. Once the transmitter determines the position of the receiver, the transmitter optionally tracks the location of the receiver to ensure that the beam is optimally positioned on the receiver during any subsequent movement of the receiver or the transmitter. The tracking function takes frequent measurements of the receiver's location and makes micro-adjustments to the beam location to keep it within the tight limits needed for optimal power transmission. By constantly tracking the receiver, the transmitter need not relocate the receiver every time it is moved, and may instead, transmit power continuously with the power beam adjusted according to the tracking function. Optional predictive software tracking algorithms may be used to more accurately track the receiver.

In another embodiment, the transmitter includes a safety feature to prevent unintended damage to property or people. In operation, the transmitter receives a portion of the beam back from the receiver and uses the reflected beam to determine and track the receiver's location. If, however, an object obstructs the beam the transmitter would detect this instantaneously because the reflected beam would necessarily also be obstructed. In such a circumstance, the transmission of power is turned off so that the beam will not cause damage to the obstructing object. This closely coupled system ensures that the beam will never be in high power mode when the reflected beam is not seen at the transmitter. This is an improvement over the prior art because beam obstruction can be detected without the need of complicated circuitry and/or another channel of communication.

In yet another embodiment, the receiver optionally comprises a modulator and the transmitter comprises a demodulator. The receiver may modulate the reflected beam in a pattern such that the reflected beam carries data. The transmitter may demodulate the reflected beam to recover the data contained therein. The data may optionally contain information that identifies and/or authenticates the receiver.

In another embodiment, the transmitter optionally comprises a modulator and demodulator, and the receiver comprises a modulator and demodulator. This embodiment allows for two way communication of data between the receiver and transmitter. And because the data is encoded on the beam and because the beam optically connects only the transmitter and receiver, the 2 way communication is secure from eavesdropping.

### 5 BRIEF DESCRIPTION OF THE DRAWINGS

FIG. 1 is an example of the transmitter and receiver used in an environment.

FIG. 2 is a schematic representation of the transmitter and receiver.

FIG. 3 is an overview of various receiver configurations.

FIG. 4 illustrates a structure that reduces the reflectance and increases the transmittance of a retro-reflector.

FIG. 5 shows a dynamic reflectance retro-reflector.

FIG. 6 illustrates the components of a type of micro-adjuster.

FIG. 7 is a graphical representation of different intensity distributions received by the transmitter from the receiver.

FIG. 8 shows a component of the receiver using a dynamic reflectance layer.

FIG. 9 is a graphical representation of modulated power intensity.

FIG. 10 illustrates two implementations of a parallel plate modulator used to encode data on the EM beam.

FIG. 11 presents a liquid crystal display modulator used to encode data on the EM beam.

FIG. 12 shows a photo detector configuration.

FIG. 13 shows a device that can modulate and demodulate the EM beam.

### 6 DETAILED DESCRIPTION OF THE INVENTION

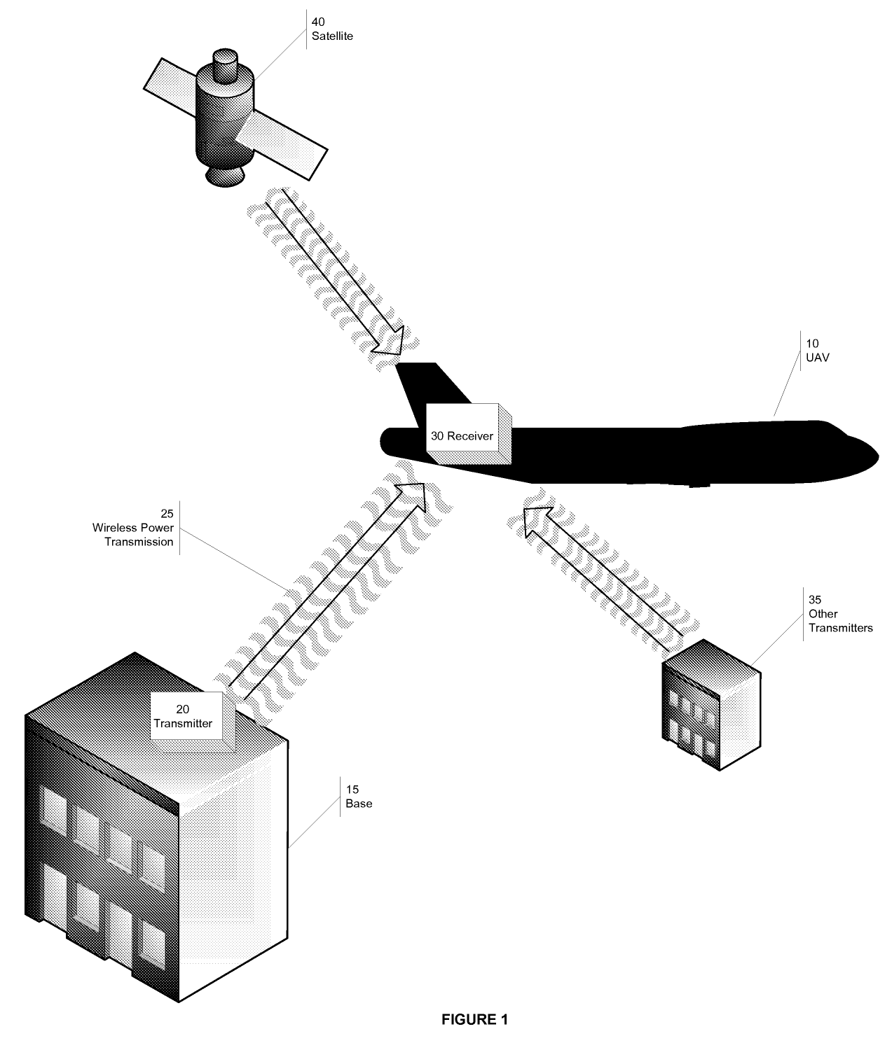

What is described below is a novel wireless energy transmitter and receiver for wireless energy and data transmission over large distances. FIG. 1 shows one example of an application of the technology described herein. An unmanned air vehicle (UAV) 10 requires significant power and, in its optimal operation, should be in the air as much as possible. Unfortunately, because UAVs 10 consume power quickly, their flying time, and consequently their range, is limited. The power transmission methods and devices described herein can be used to transmit energy efficiently and effectively to the UAV 10, dramatically increasing the UAV's range and flying time. Specifically, the base 15 contains a transmitter 20 that transmits wireless power 25 to a receiver 30 located on the UAV 10, allowing the vehicle to have a much longer surveillance runs. Other strategically positioned power transmitters 35 could be placed near the UAV's 10 surveillance area to further increase the UAV's 10 range and minimize the down time for power recharging. It is also possible to position transmitters on satellites 40 or other types of vehicles (aerial, terrestrial or aquatic), again extending the distance the UAV 10 can travel from the base 15. The UAV application just described is given to illustrate the use of the technology in a particular environment. It represents just one example of how the technology described herein can be used and, as such, should not be read to limit the scope of the claims that follow in any way.

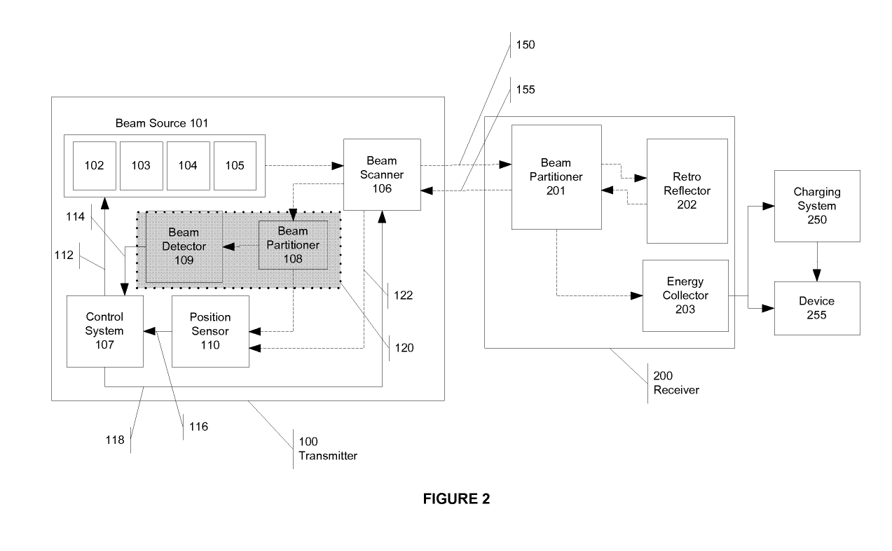

FIG. 2 illustrates the basic structures contained in the transmitter 100 and receiver 200. The dashed arrows represent optical connections, while the solid lines represent electrical connections. In overview of the transmitter 100 comprises a beam source 101 which may contain one or more of the following beam modifying components: an electromagnetic generator 102 that provides the electromagnetic (EM) beam, collimating optics 103 that collimate the EM beam when necessary, an intensity control mechanism 104 to switch between a low or high power EM beam (in another embodiment, this function can be performed by the beam generator 102), and beam conditioning optics 105 that optimize the EM beam width and shape at the receiver 200 for optimal energy transfer. The transmitter 100 also may include a beam scanner 106 to direct the EM beam 150. The beam scanner 106 may comprise both a macro adjuster and micro adjuster. The transmitter 100 may also include a beam partitioner 108 that splits the reflected EM beam 155 between the beam detector 109 and the position sensor 110. The beam detector 109 detects the presence of the reflected EM beam 155 that is reflected by the receiver 200. The position sensor 110, working in conjunction with the control system 107, locates and tracks the reflected EM beam 155 from the receiver 200 and analyzes the reflected EM beam's 155 shape and intensity. While this embodiment employs both a beam detector 109 and a position sensor 110, the functions of both of these structures can be performed by the position sensor 110. In this case, the beam partitioner 108 and the beam detector 109 would not be needed, so the optical pathway of the reflected beam 155 would follow the arrow 122. The components shown in the shadowed box 120 are redundant, and may optionally be used as an additional safety measure. Finally, the transmitter 100 also includes the control system 107 that receives a position sensor signal 116 that includes information regarding characteristics of the reflected beam 155, and may also receives a beam detector signal 114 from the beam detector 109 (if this component is used) that also contains information regarding the reflected beam 155. The control system 107 processes one or both of these signals to generate a beam modification signal 112 that controls the beam source 101, which in turn modifies the EM beam 150. The control system may also send a beam adjustment signal 118 (based on one or both of the signals 114 and 116) to the beam scanner 106 to control the direction of the EM beam 150. In an alternate embodiment, the transmitter contains a second beam partitioner and a second beam detector so that the EM beam 150 can be imaged. Specifically, the beam partitioner could be placed in the optical pathway of the beam source 101 such that it partitions the EM beam 150 with one portion hitting the second beam detector and another portion transmitted to the receiver 200. In this embodiment, the imaging of the EM 150 beam can assist in tracking the receiver 200 and in confirming that the reflected beam 155 detected by the transmitter 100 was indeed sent by the transmitter 100.

An overview of the receiver 200 comprises a beam partitioner 201 that splits the received incoming EM beam 150 between the retro-reflector 202 and the energy collector 203. The retro-reflector 202 reflects a portion of the incoming EM beam 150 back to the transmitter 100 (resulting in a reflected EM beam 155), and the energy collector 203 collects and converts the incoming EM beam's 150 electromagnetic energy into electrical energy, suitable for use by electronic devices 255. The receiver 200 may optionally include a charging system 250 that takes the power from the energy collector 203 to charge a power reserve such as a battery or a capacitor.

Now that an overview of the various components of the transmitter 100 and receiver 200 has been presented, a detailed description of those components follows. In operation, the receiver 200 receives an incoming EM beam 150 comprised of electromagnetic energy. The receiver 200 reflects a portion of the incoming EM beam 150 back to the transmitter 100, so that the transmitter can more accurately direct and focus the incoming EM beam 150 through the use of the beam scanner 106 and beam source 101. Once the receiver 200 receives the incoming EM beam 150, several embodiments may be employed to receive the most amount of power at the energy collector 203, while also providing a reflected EM beam 155 to the transmitter 100 for positioning, focusing and tracking. Three such embodiments may include using: A) A traditional beam splitter; B) a partially transmissive single corner cube retro-reflector; and C) an array of corner cube retro-reflectors. All of these embodiments may be made partially transmitting or segmented by regions.

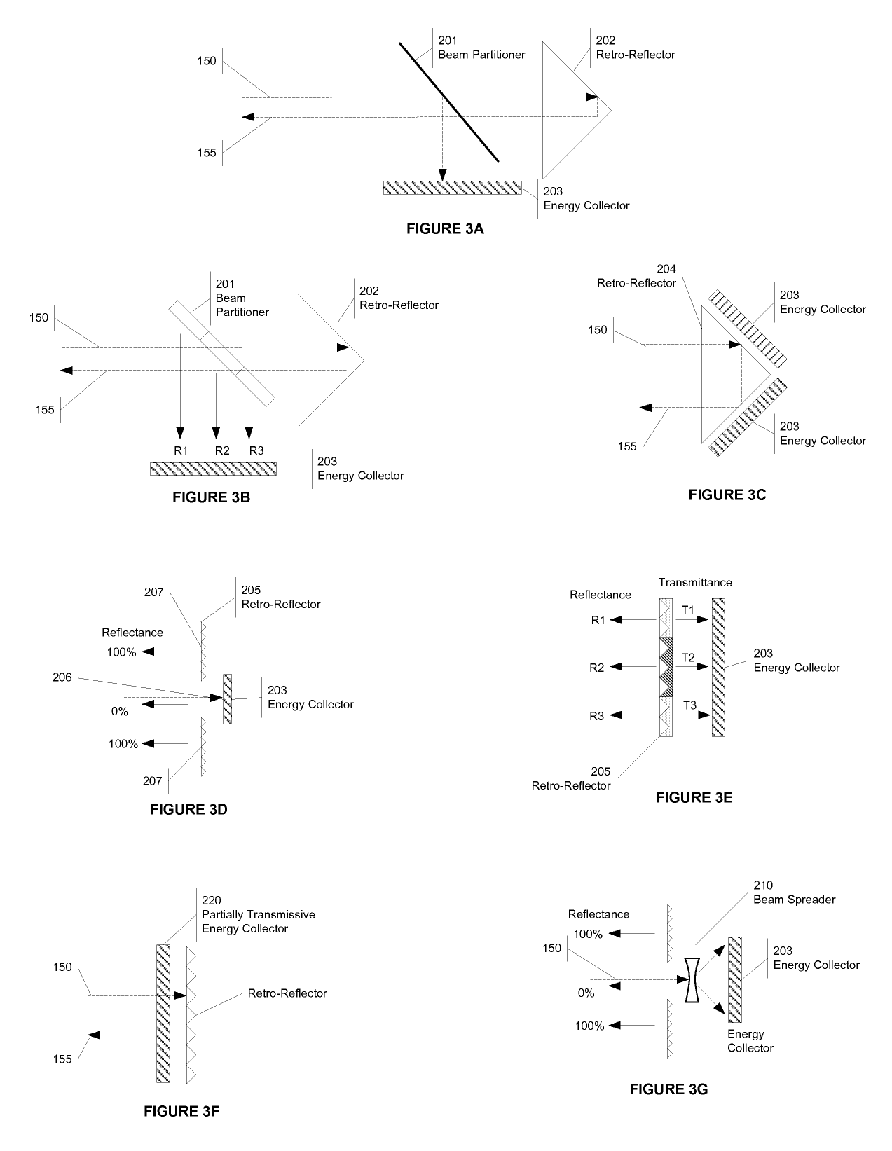

The first embodiment is shown in FIG. 3A. The beam partitioner 201 partitions the incoming EM beam 150. In this embodiment, the beam partitioner 201 may be a partially transmitting mirror. A portion of the incoming EM beam 150 is directed to the retro-reflector 202 and a portion is directed to the energy collector 203. A retro-reflector 202 is commonly used in optics and has the desirable characteristic that an incoming EM beam arriving at the retro-reflector 202 is reflected parallel to the incoming EM beam. Thus, the retro-reflector 202 need not be perpendicular to the incoming EM beam to completely reflect it. This means that the receiver 200 need not be perpendicular to the incoming EM beam 150 to provide a reflected EM beam 155 that may be effectively used by the transmitter 100. Although a retro-reflector may be advantageous, it would be apparent to those skilled in the art that various types of reflectors may be used including, but not limited to, mirrors, prisms, polished surfaces, paints, retro-reflective beads, glass and retro-reflective paints. It would also be apparent to one skilled in the art that depending on the application, the reflector may be constructed from various materials including, but not limited to, hard materials (glass, plastic, polymers, etc.), semi-hard materials (plastics, polymers, gels, etc.), soft materials (glass, plastic, polymers, etc.), reflecting liquids, and nanomaterials.

The retro-reflector 202 may be constructed of a single corner cube or an array of corner cubes. As shown in FIG. 3B, by coating the beam partitioner 201 with know materials, the beam partitioner 201 may selectively partition the incoming EM beam 150 into R1, R2 and R3. For example, if the transmitter 100 transmits a multi-frequency incoming EM beam 150, a properly coated beam partitioner 201 could be made to separate frequencies; e.g. one for tracking (i.e., the EM beam that is reflected), one for power absorption by the energy collector 203, and one for data transmission/reception. A dichroic mirror may be used as the beam partitioner 201 to selectively split the incoming EM beam 150.

In another embodiment shown in FIG. 3C, the beam partitioner is a single corner cube retro-reflector 204 that is located in front of one or more energy collectors 203. Multiple energy collectors 203 can be used by placing them parallel to the sides of the corner cube retro-reflector 204. This allows the maximum amount of energy to be collected from the various faces of the cube-retro-reflector 204. The reflectivity of each face of the retro-reflector 204 can be made to optimize the retro-reflected component, and the transmitted component to the energy collector 203. The portion of the EM beam 150 that is not retro-reflected is transmitted through the retro-reflector 204 to the energy collector 203, in this way the retro-reflector 204 partitions the EM beam 150.

In yet another embodiment shown in FIGS. 3D and 3E, the beam partitioner is an array of single corner cube retro-reflector 205 that is located in front of one or more energy collectors 203. Using an array of corner cubes, it is possible for the depth of the corner cubes in the array to be substantially smaller than that of a single corner cube, permitting a more compact setup. While the energy collector 203 is shown to be behind the retro-reflectors, as shown in FIG. 3F it is possible to construct a partially transmissive energy collector 220 that allows a portion of the incoming EM beam 150 to cross the energy collector to reach the retro-reflector, and the reflected EM beam 155 could then pass back through the energy collector. Also, referring to FIG. 3G, it may be advantageous to add optics such as a beam spreader 210 (such as a suitable wide angle lens like a negative lens or fish eye lens), which may be placed in front of the energy collector 203, to optimize energy absorption by spreading the EM beam 150 over the full area of the energy collector 203. Alternatively, the beam spreader 210 may be placed in front of the reflector to increase the EM beam acceptance angle of the receiver 200. Finally, two or more beam spreader 210 may be used to both optimize the energy absorption and increase the acceptance angle.

The retro-reflector 205 may have a uniform reflectance, but in a preferred embodiment, the retro-reflector 205 is not uniformly reflective; rather it has at least two regions that differ from each other in reflectance. The variation in reflectance can be achieved by cutting out a portion 206 of the retro-reflector 205 as shown in FIG. 3D. The retro-reflector 205 has a region 207 with a reflectance of 100% and the cut-out region 206 with a reflectance of 0%. Alternatively, FIG. 3E does not contain a cutout, but the retro-reflector 205 nevertheless contains a three regions of reflectance R3, R2 and R1, and three corresponding regions of transmittance T3, T2 and T1. The advantage to having some transmittance is that the energy collector 203 can collect more energy if more of the incoming EM beam 150 is allowed to reach it. Again the retro-reflector 205 partitions the EM beam 150.

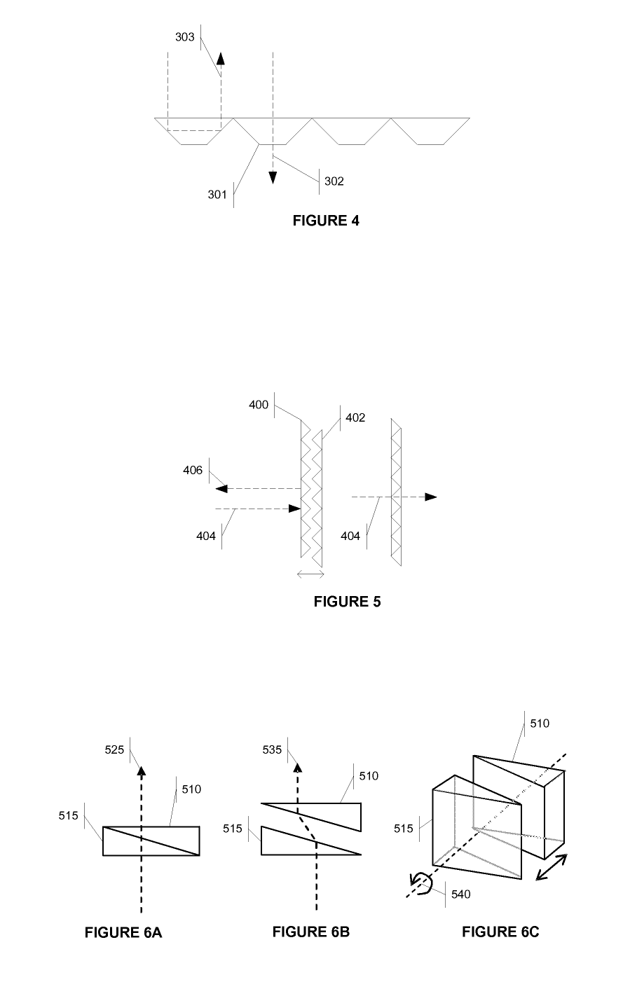

The variation in reflectance can be achieved by various methods. As discussed above, the retro-reflector may have a cutout or punched holes. Also, depositing different optical materials on the backside of the retro-reflector allows part of the incoming beam to be reflected and part to pass through to the energy collector. A mask can be applied to the front side of the retro-reflector. Another method to change the reflectance is shown in FIG. 4. The geometry of the corner cube retro-reflector can also be modified to vary the amount of light reflected. For example, the corner of the retro-reflector can be made blunt 301 causing some of the incoming EM beam to pass through 302 while some is retro-reflected 303. The retro-reflector can be made blunt by cutting, polishing or using a mold with the corners blunted.

Another method for varying reflectance is shown in FIG. 5. Two corner cube retro-reflector arrays 400 and 402 are arranged parallel to each other with their backsides facing each other (i.e., the side opposite to the flat face of the retro-reflector). When the arrays 400 and 402 are separated by a distance, the incoming EM beam 404 is retro-reflected 406. However, when the arrays 400 and 402 mate then the incoming EM beam 404 is allowed to pass through. The distance between the arrays 400 and 402 determines the amount of reflectance. The non-uniform reflectance assists the transmitter in accurately determining the position of the receiver as discussed below. It may be advantageous to vary the materials used to construct the retro-reflector arrays 400 and 402. For example, if one of the arrays is made of a soft material, it may ensure a better fit when the arrays are brought together, ensuring a more precise operation. It would also be apparent to one skilled in the art that depending on the application, the retro-reflector arrays 400 and 402 (in addition to all the retro-reflectors described herein) may be constructed from various materials including, but not limited to, hard materials (glass, plastic, polymers, etc.), semi-hard materials (plastics, polymers, gels, etc.), soft materials (glass, plastic, polymers, etc.), reflecting liquids, and nanomaterials.

Regardless of how the beam partitioner is implemented, it is advantageous to have a large portion of the incoming EM beam 150 directed to the energy collector 203. The energy collector 203 may be a photo cell or array of photo cells that convert(s) the incoming EM beam 150 energy to electricity and, may also optionally include power conditioning electronics that transforms the converted electricity into a usable power source that can be used by a battery charging system 250 or power consuming device 255.

In all implementations of the beam partitioner described above, a portion of the incoming EM beam 150 is reflected back to the transmitter 100. Because the incoming EM beam 150 is reflected, the receiver 200 is passive—i.e., it does not require power for the transmitter 100 to locate or track it. This is advantageous because, unlike existing systems, the receiver 200 does not require an independent source of power before receiving wireless power transmissions. In addition, the passive nature of the receiver 200 reduces complexity and provides faster feedback compared to existing systems which require active feedback via a second communication channel.

Turning now to the transmitter 100 shown in FIG. 2. In one embodiment, the control system 107 is comprised of a micro processor running tracking and control software. The control system 107 controls the beam source 101 and beam scanner 106 with information processed from the beam modification signal 112 and the beam adjustment signal 118. Additionally, the control system 107 may have a standard interface, such as a USB port, for monitoring or controlling the transmitter 100 remotely. When the system begins the process of locating the receiver 200, the control system 107 turns on the electromagnetic beam generator 102. The source of the electromagnetic waves can include EM sources such as thermal, laser, gas discharge, arc, or other electromagnetic sources know to those skilled in the art. To begin locating the receiver 200, the control system 107 sends instructions to the intensity control 104 which sets the EM beam to low power using shutters, multiple beams, or controlling the intensity of the source directly. The EM beam 150 emitted from the transmitter 100 while in the low power mode is set to an intensity that is safe for the environment in which it is being used. The control system 107 may also direct the beam conditioning optics 105 of the transmitter 100 to defocus the EM beam 150. Defocusing allows the receiver 200 to be located more rapidly because the EM beam 150 is spread over a larger volume of space, so the reflected EM beam 155 from the receiver can be achieved faster—often in a single pass of the EM beam 150.

As already described, an embodiment of the present system uses a low powered and defocused EM beam 150 to scan a large area for the receiver 200. The control system 107 instructs the beam scanner 106 to direct the EM beam 150 over a volume of space in a predetermined manner. The beam scanner 106 may comprise two types of beam adjusters that manipulate the location of the EM beam 150—i.e., a macro adjuster and a micro adjuster. The macro adjuster is comprised of mirrors, rotating surface, MEMS, or other structures known to those in the art, and is used to scan a large volume of space. The micro adjuster scans a smaller area with a higher level of precision. During the initial location of the receiver, the macro adjuster will direct the EM beam 150. Once the transmitter 100 detects the receiver 200 by detecting a reflected EM beam 155 at the position sensor 110, then the position sensor 110 sends a position sensor signal 116 to the control system 107. The control system 107 processes this signal and sends a beam modification signal 112 to the beam source 101 to focus the EM beam 150 (and optionally to increase the beam's intensity). At this point, the control system 107 may also send a beam adjustment signal 118 to the beam scanner 106 to direct the EM beam 150 to the region where the reflected beam 155 was detected.

Both the macro and micro adjusters work to hone the EM beam 150 on the receiver 200, but for the small changes to the location of the EM beam 150, the micro adjuster may be used more heavily. The combination of macro and micro adjuster allows rapid and efficient location of the receiver 200. Traditional beam scanners, such as a rotating mirror, vary based on their precision and angular resolution and typically, a high precision traditional beam scanner with fine angular resolution is more expensive and slow for covering a large area. In addition, as the distance between the transmitter 100 and the receiver 200 increases, the beam scanner's level of precision decreases, making it very difficult to hone in on a receiver 200 to impart a focused high powered EM beam 150. Therefore, if the receiver 200 is a substantial distance from the transmitter 100, it can be challenging for a traditional beam scanner to accurately place a beam on a receiver. The micro adjuster illustrated in FIG. 6 overcomes the problems of traditional beam scanners by allowing translational (as opposed to angular) beam scanning. Translational beam scanning allows for the adjustment of the EM beam by small amounts regardless of the distance between the transmitter and receiver. For example, a translation of the EM beam by 10 millimeters will move the beam by 10 millimeters whether the receiver is 10 meters or 1000 meters away. The micro adjuster consists of two prisms (510 and 515) oriented to face each other. The distance between the prisms can modified using simple electro-mechanics. When there is no separation between the prisms (FIG. 6A) the EM beam 525 passes straight through the micro adjuster without being translated. If there is a separation between the prisms (FIG. 6B), the beam is translated 535 proportionately to the distance between prisms, the further the distance between prisms, the greater the translation in the beam. Because this is a translation rather than an angular modification, accurate micro adjustments can be made regardless of the distance between the transmitter and receiver. To achieve translation of the beam in all directions the prisms can be rotated 540 (FIG. 6C) as a single unit using electro-mechanics. Translation in all directions can also be achieved by using two sets of prisms oriented perpendicular to each other providing translation in both the X and Y axes.

Depending on the application, the micro adjuster may also use fine angular beam scanning to properly position the EM beam 150 on the receiver 200. In one implementation, using negative and positive lenses can provide very fine angular adjustments (more fine than traditional beam scanners such as a rotating mirror) to the EM beam 150, adjustments that would not be possible with the macro-adjuster.

As the transmitter 100 works to locate the receiver, the beam scanner 106 directs a low powered defocused the EM beam 150 over a volume of space in a predetermined manner. During this coarse-macro location step, the EM beam 150 is low powered and defocused (i.e., spread) such that it will not cause interference or damage to property or people. The transmitter 100 detects the location of the receiver 200 when it detects the reflected beam 155 reflected from the receiver 200. The portion of the beam that is reflected 155 is returned to the transmitter 100 and directed to the transmitter's beam partitioner 108. The beam partitioner 108 can be implemented in a variety of ways, however, in the preferred embodiment of the beam partitioner 108 is implemented using a standard beam splitter. The beam partitioner 108 directs a portion of the reflected beam 155 to the beam detector 109 and a portion to the position sensor 110. The position sensor 110 is comprised of a standard imaging array such as a CCD (Charge Coupled Device), CMOS (Complementary metal oxide semiconductor) image sensor, or array of photo detectors. The position sensor 110 is coupled to the control system 107 and supplies information to the control system 107, which the control system 107 can use to control the various components of the beam source 101 and the beam scanner 106. As the transmitter 100 is scanning for the location of the receiver 200, the control system 107, in conjunction with the position sensor 110, monitors the intensity of reflected EM beam 155. When a significant increase in the intensity of reflected EM 155 beam is detected, the control system 107 (based on the position sensor signal 116) signals the conditioning optics 105 (through the beam modification signal 112) to focus the EM beam 150 and sets the beam scanner 106 (through the beam adjustment signal 118) to scan a smaller area localized around the position where the beam intensity was detected. As the area being scanned decreases, the micro adjusters will play a larger role in pin pointing the EM beam 150 on the receiver 200. This process is repeated until the EM beam 150 is optimally positioned on the receiver 200. One such instance of the EM beam 150 being placed optimally on the receiver 200 may occur when the EM beam 150 waist is narrower than the retro-reflector 202.

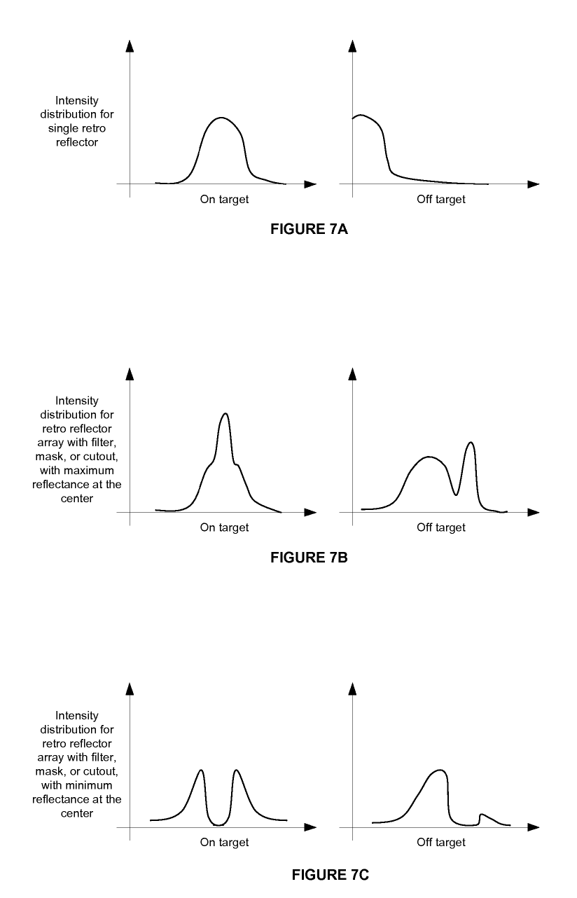

The control system 107 may use the intensity distribution of the reflected EM beam 155 to further hone the position of the EM beam 150. Referring to FIG. 7A, the intensity distribution of a single cube retro-reflector may look like a bell curve. If the EM beam 150 is not directed precisely on the retro-reflector (right side of FIG. 7A), then only a portion of the EM beam 150 is reflected back to the transmitter. From this energy distribution, the control system 107 can direct the beam scanner 106 to reposition the EM beam 150 until the control system 107 detects an energy distribution that is on target (left side of FIG. 7A). When using an array of retro-reflectors, the control system 107 may use the non-uniform reflectance of the retro-reflector to more accurately position the EM beam 150 on the receiver 200. FIG. 7B illustrates an energy distribution for a non-uniform reflectance where the center of the reflector reflects more than the outside of the reflector. When the transmitter 100 first detects the receiver 200, the transmitter may detect the intensity distribution shown on the right side of FIG. 7B. The control system 107 then directs the beam scanner 106 to more accurately position the EM beam 150 on the receiver 200, until the control system 107 detects an energy distribution that is on target (left side of FIG. 7B). FIG. 7C illustrates an energy distribution for a non-uniform reflectance where the center of the reflector reflects less than the outside of the reflector. Again the control system 107 uses the detected energy distribution to hone the EM beam 150. For the fine EM beam honing required to achieve the on-target energy distribution, the control system 107 may use both the macro and micro adjusters contained in the beam scanner 106. However, as the reflected EM beam 155 begins to approach the on-target energy distribution, it is likely that only slight movements to the EM beam 150 are necessary, so the control system 107 would rely more heavily (and in some cases, exclusively) on the micro-adjusters. Once the control system 107 confirms that EM beam 150 is on-target (by processing the position sensor signal 116), it sends a beam modification signal 112 to the intensity control 104 to turn the EM beam 150 to high power to maximize energy transmission. The control system 107 may also send control signals to the position sensor 110 and beam detector 109 to modify parameters such as sensor gain.

The intensity distribution of the reflected EM beam 155 can also be used to make certain that the expected receiver 200 has been located rather than another reflective surface. There are several ways for the receiver 200 to add a “signature” to the reflective EM beam 155 if desired including: diffraction, refraction, or reflection (accomplished by using gratings, wire arrays, etch plates, etc.), all of which would yield an expected (and possibly unique) intensity distribution, or by modulating the beam spatially or temporally. It should be noted that the energy distributions in FIGS. 7A-7C are shown in one dimension for simplicity. In practice the energy distribution would be in two dimensions, but it would be clear to those in the art from the disclosure herein how to implement an EM honing method for two dimensional energy distributions.

Once the receiver 200 has been located and the high power EM beam 150 is being transmitted, the process of tracking the receiver 200 begins. The process of tracking is similar to the process described above to locate the receiver 200. If the receiver 200 (or the transmitter 100) is moved from its original position, the intensity distribution of the reflected EM beam 155 will change and the control system 107 will detect this through the position sensor 110 and the corresponding position sensor signal 116. The control system 107 then directs the beam scanner 106 (through the beam adjustment signal 118) to make any adjustments to the position of the EM beam 150 so that the on-target intensity distribution is once again achieved. In addition, the control system 107 continually adjusts the conditioning optics 105 (through the beam modification signal 112) to maximize energy transfer based on the position sensor signal 116. Predictive tracking software algorithms can be used to optimize the tracking of the receiver. Algorithms such as the Kalman filter or particle filter can be used for this purpose.

It is important to note that the dynamic reflectivity illustrated in FIG. 5 and described above may be used by the system to more quickly locate and track the receiver 200, and to more efficiently transmit energy. For example, the parallel retro-reflector arrays 400 and 402 would be separated during location and/or tracking, yielding an intense reflected EM beam 406 back to the transmitter 100. During power transmission, however, the retro-reflector arrays 400 and 402 would be mated to allow most of the EM beam 404 to hit the energy collector. The receiver 200 could control this by monitoring the energy intensity hitting the retro-reflector arrays—i.e., low intensity means the transmitter 100 is locating the receiver 200, thus the arrays are separated; while high intensity means the transmitter 100 and located the receiver 200, so the arrays are mated. This configuration has the benefit of reducing the reflectance during high energy transmission, thus increasing the energy transmission efficiency of the system.

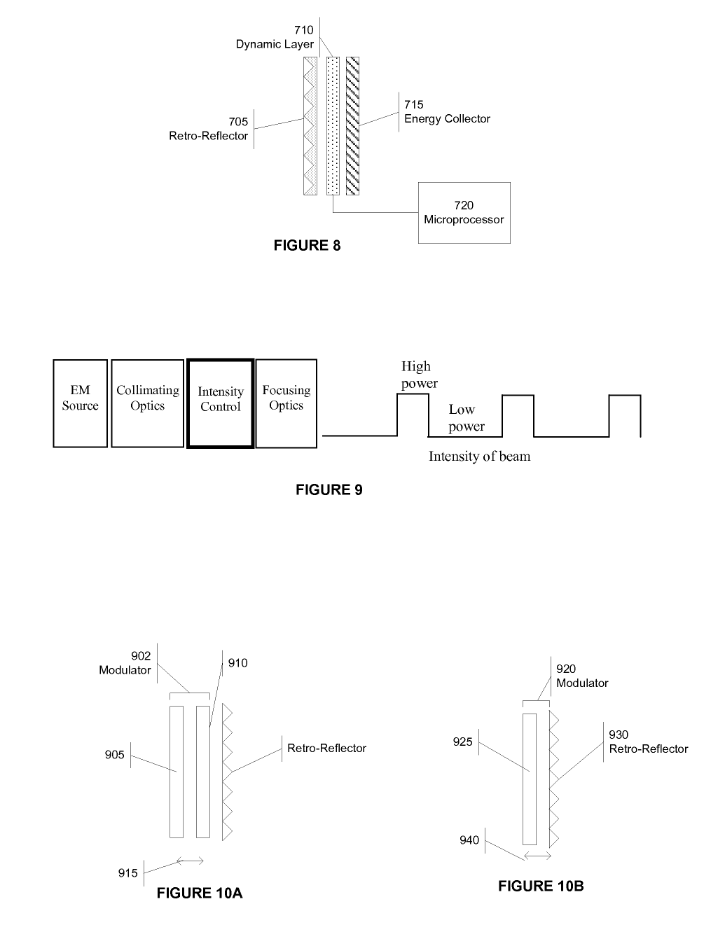

Referring to FIG. 8, a dynamically reflective layer may also be used to optimize location, tracking and energy transmission. Specifically, a dynamic layer 710 may be sandwiched between a retro-reflector 705 and the energy collector 715. During tracking, the dynamic layer 710 will have a high reflectance. But during energy transmission, the layer 710 would have a low reflectance, allowing much of the incoming EM beam to reach the energy collector 715. Again, the receiver 200 would monitor the incoming EM beam to determine how to control the layer's 710 reflectance, and that control would be performed by a microprocessor 720. Alternatively, the dynamic layer 710 can also be placed in front of the retro-reflector 705.

When the transmitter 100 is in the initial coarse-macro location mode, the EM beam 150 is low powered and de-focused, which renders it safe. However, once the EM beam 150 is focused, its power intensity increases and once the high power beam is activated the power intensity further increases. Thus, as an additional safety feature, the system may optionally have a beam detector 109 used to shut of the beam source 101. A portion of the reflected beam 155 is directed to the beam detector 109 by the beam partitioner 108, which provides a signal to the control system 107 that indicates the presence of the reflected beam 155 and serves as a rapid safety shutdown mechanism. The beam detector 109 may be comprised of a photo detector and conditioning optics, if necessary. When the beam detector 109 senses a reflected beam 155 with the expected intensity a beam detector signal 114 is sent to the control system 107. If at any point the beam detector 109 ceases to detect the reflected beam 155, the beam detector signal 114 will alert the control system 107, which will send a beam modification signal 112 to shut down the transmission of the high power EM beam 150. Optimally, this feedback loop should not require software or a microprocessor, which would yield a rapid, highly effective, tight and dependable feedback loop, unlike previous wireless power transmission systems (see e.g. U.S. Pat. No. 2004/0227057 A1). Also unlike previous systems, this feedback loop does not require a second transmission channel. This closely coupled feedback system ensures that at no time will the beam source 101 be in high powered mode when the reflected EM beam 155 is not seen at the beam detector 109. The position sensor 110 is continually analyzing the intensity distribution of the reflected beam 155. If at any time the intensity distribution does not match the expected distribution, the control system 107 can direct the EM beam source 101 through a beam modification signal 112 to power down. Thus, the position sensor 110 can serve the dual function of positioning the EM beam 150 and reflected beam 155 detection, obviating the need for a separate beam detector 109. The beam detector 109 is used as a redundant, although not necessary, safety system. If this redundancy is not used, then there would be no need for the beam partitioner 108 or the beam detector 109 (see shadowed box 120 that illustrates the redundant safety features). Should the reflected beam 155 be partially occluded it is possible, in some instances, to continue to track the receiver 200 using the low power beam. If the reflected beam 155 is completely occluded, the control system 107 would direct the beam source 101 to power down and defocus the EM beam 150. The control system 107 would then begin the process of coarse-macro location of the receiver 200 as described above; however because the transmitter 100 knows the last location of the receiver 200, it may direct the EM beam 150 to the region surrounding the last know location of the receiver 200. This could allow the transmitter 100 to more quickly locate and track the receiver 200.

Additional safety mechanisms can be implemented if the system application warrants. For example, as shown in FIG. 9 the EM beam 150 can be modulated (from high power to low power) to provide additional safety. This will minimize the energy delivered to an object that blocks the beam. This intensity control may be implemented using shutters, multiple beams, or controlling the intensity of the source directly. If temporal modulation is used, the low intensity periods can be use for continuous tracking of the receiver. The modulation cycle will be a function of the application. Additionally, the transmitter 100 can add a signature to the EM beam 150 that is transmitted by modulating the EM beam 150. When the position sensor 110 analyzes the reflected EM beam 155 it will verify that the transmitted 150 and reflected EM beams 155 are from the same source and not a random source. Similarly, the receiver 200 may modulate the reflected EM beam 155 which confirms that the receiver 200 is the proper recipient of the EM beam 150.

Depending on the specific needs and capabilities of the application, the source of the electromagnetic waves can include electromagnetic sources such as thermal, laser, gas discharge, arc, or other. The wavelength range could be chosen within the visible or non-visible spectrum depending on the application. When a non-collimated electromagnetic source is being used, such as thermal, gas discharge, or arc, a concentrator and collimating optics may be used. Non-laser sources are attractive because of their simplicity, lower cost, and higher efficiency. Available technology can concentrate the emission of these sources to be practical for the systems described in this invention.

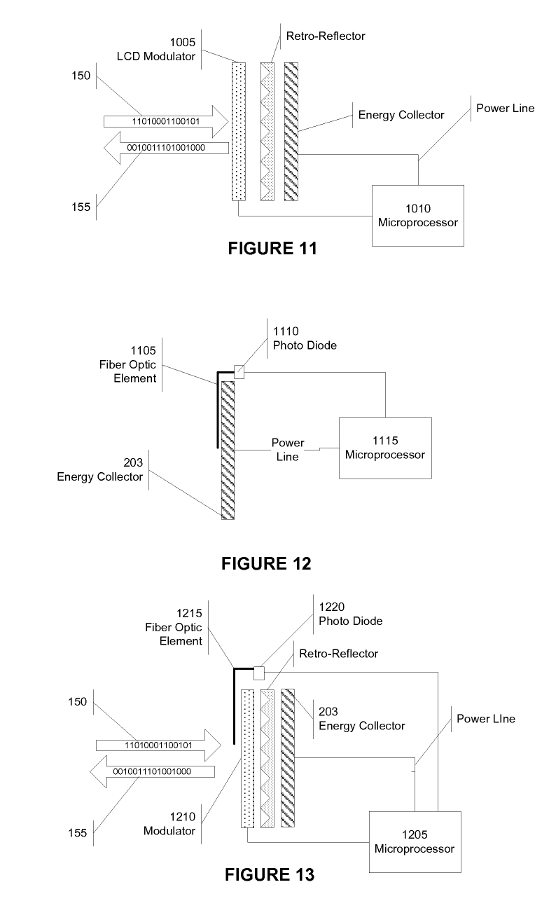

One way or full duplex data communications can also optionally be implemented in the current energy transmission system. The transmitter 100 can add data to the beam by modulating the outgoing EM beam 150 temporally or spatially. The beam partitioner 201 of the receiver 200 can divert some of the energy to a photo detector that will read the incoming communications data. In order to encode data on the reflected EM beam 155, the beam 155 can be modulated by modulating the beam 155 spatially or temporally (using acoustic, non-linear crystal modulators, etc.) FIGS. 10A and 10B illustrate embodiments of a parallel surface modulator. Specifically in FIG. 10A, the modulator 902 comprises two parallel plates (905 and 910) separated by distance 915. By varying the distance 915, the modulator 902 can be transparent or opaque. The distance between the plates (905 and 910) can modified using simple electro-mechanics. Thus, the reflected EM beam 155 could be encoded with data by modulating the reflected EM beam 155 with the modulator 902. FIG. 10B illustrates another modulator configuration. Here the modulator 920 is comprised of a plate 925 and the retro-reflector 930 separated by a distance 940. To optimize the operation of this modulator 920, it may be advantageous to apply an appropriate coating to the retro-reflector 930. Again, varying the distance 940 causes the modulator to be transparent or opaque, which, in turn, allows the encoding of data on the reflected EM beam 155. The two modulator structures shown in FIGS. 10A and 10B are similar to a Fabry-Perot interferometer. FIG. 11 shows yet another modulator embodiment. Here, the modulator 1005 is a dynamically translucent liquid crystal display (LCD); however other optical modulators that are not based on LCD technology can be used as well. A microprocessor 1010 may vary the voltage applied to the LCD modulator 1005, causing the LCD modulator to modulate between being transparent and opaque, which again allows the encoding of data on the reflected EM beam 155.

When the reflected EM beam 155 is received at the transmitter 100, the beam detector 109 and/or position sensor 110 can be used to extract the data from the reflected beam 155. On the receiver 200 side, the receiver 200 can monitor the incoming EM beam 150 for a modulation using the energy collector 203, and could decode the modulation to extract the data. In another embodiment shown in FIG. 12, a fiber optic element 1105 can be place in front of the energy collector 203. The element 1105 is connected to a photo-diode 1110, which converts the optical signal into an electrical signal that is feed into a microprocessor 1115. The microprocessor 1115 can decode the modulated signal. While the fiber optic element 1105 is shown in a single location in front of the retro-reflector, depending on the application the location can be changed and/or multiple locations can be used.

In the embodiment shown in FIG. 13 various elements of FIGS. 11 and 12 are combined to form a device can modulate the reflected EM beam 155 and demodulate the EM beam 150. The microprocessor 1205 controls the modulator 1210 to modulate the reflected EM beam 155 as described above. The microprocessor can also demodulate the EM beam 150 using the fiber optic element 1215 connected to a photo-diode 1220 which converts the optical signal into an electrical signal. In this embodiment, the photo-diode 1220 may read a modulated signal that contains both the modulation encoded by the transmitter 100 (i.e., on the EM beam 150) and the modulation encoded by the receiver 200. To parse out the modulation pattern from the transmitted 100, the receiver 200 should subtract out its modulation pattern from the pattern detected by the photo-diode 1220. This can be achieved using basic logic operators and/or a number of multiplexing techniques. Alternatively, a beam partitioner may be used to partition a portion of the EM 150 to the demodulator, which would allow the photo-diode to read only the EM beam 150 as modulated by the transmitter.

In two-way communication, the transmitter 100 and receiver 200 can encode data by modulating the EM beam 150 or the reflected EM beam 155 as discussed above. To decode the data, the receiver 200 may only look at the incoming modulated EM beam 150. The receiver 200 can also further modulate the reflected EM beam 155 to encode data that is to be received by the transmitter 100. In this case, however, the transmitter will receive a reflected EM beam 150 that has both the data the transmitter 100 originally encoded and the data newly encoded by the receiver 200. As discussed above, the transmitter 100 should subtract out its modulation pattern from the reflected EM beam 155 modulation pattern, which can be achieved using basic logic operators and/or a number of multiplexing techniques.

An additional benefit to one or two-way communication using the aforementioned system is that it is a secure communication. Outside eavesdroppers will not see the EM beam, therefore they cannot intercept the encoded data. A secure two-way data transmission system could operate as follows. The transmitter 100 could locate and find a receiver 200. Using public key/private key authentication, the receiver 200 would send an authenticating key to the transmitter 100. At this point a private line of communication has been established. Also, given that the transmitter 100 knows general location of the receiver 200, it is very unlikely that a rogue receiver would be in the area and would intercept the EM beam transmission. And even if it were to intercept the transmission, it would be even more unlikely that the rogue receiver would know the proper authentication key.

Depending on the needs of the application, the receiver can be augmented in several ways. For example, more than one energy collector can be used to maximize energy collection by recovering energy that would otherwise be lost by the beam partitioner. In some instances, it may be desirable to have one receiver with multiple retro-reflectors with dedicated functions, such as one for passive communication with the transmitter, a second for power collection, and a third to send data by temporally modulating the returned beam. In this configuration, once the retro-reflectors have been located, power can be transmitted to each of the retro-reflectors in a pre-determined manner.

Several beneficial applications exist for the technology described herein. For example, the system may be used for UAVs as described above. In another application, the power transmission methods and devices described herein could be used to transmit power to remote areas. Specifically, two sets of power transmitters and receivers can be used in the following manner. A terrestrial-based transmitter transmits an EM beam to a satellite, which receives the power and transmits a power beam to a receiver, which may be stationary or mobile (i.e., a UAV). The benefit of the two-step power transmission is that it can be used to transmit power to a position that would otherwise be impossible given the EM beam's line of sight limitation. Of course, the two-step power transmission scheme can be made more complicated by adding more sets of power transmitters and receivers, resulting in a three-step (or more) power transmission scheme. The additional steps may be necessary to transmit power, for example, to the opposite side of the world.

In yet another application, the technology described herein can be used for long distance identification, much like RFID but at much longer distances. Once the receiver is located by the transmitted, the receiver can modulate the reflected beam to encode identification data. The transmitter can then decode the modulated reflected beam and identify the receiver. This application is particularly useful in identifying vehicles at a checkpoint. The vehicle can be checked at a distance of several hundred meters, and if the vehicle is not properly authenticated, it may be turned away before it comes too close to the checkpoint. Or this could be useful in identifying vehicles for payment of highway and parking tolls. Having described the system in detail and by reference to several preferred embodiments thereof, it will be apparent that modifications and variations are possible without departing from the scope of the invention defined in the following claims. Moreover, the applicant expressly does not intend that the following claims “and the embodiments in the specification to be strictly coextensive.” Phillips v. AHW Corp., 415 F.3d 1303, 1323 (Fed. Cir. 2005) (en banc).

1. A wireless power transmission system comprising:
a transmitter comprising a control system that controls a beam source that generates a beam, and controls a beam scanner that directs the position of the beam;
a receiver comprising a beam partitioner optically connected to a reflector and an energy collector, where the beam partitioner receives the beam and partitions the beam so that a first part of the beam hits the reflector and becomes a reflected beam, and a second part of the beam hits the energy collector;
a position sensor that receives the reflected beam and sends to the control system a position sensor signal that contains characteristics of the reflected beam; and
the control system further performs the steps of:
receiving the position sensor signal;
based on position sensor signal, sending a beam modification signal to the beam source commanding the beam source to modify the beam; and
based on position sensor signal, sending a beam adjustment signal to the beam scanner to adjust the position of the beam.

2. The wireless power transmission system of claim 1 wherein the receiver is passive such that it receives substantially all its power from the beam.

3. The wireless power transmission system of claim 1 wherein the beam scanner comprises at least a macro adjuster and a micro adjuster.

4. The wireless power transmission system of claim 3 wherein the micro adjuster comprises a first set of prisms oriented to face each other, wherein disposed between the first set of prisms is a distance that can be varied, and the distance determines the amount of translation of the beam through the first set of prisms.

5. The wireless power transmission system of claim 4 wherein the beam travels along an axis, and wherein the first set of prisms is rotated along the axis to allow the micro adjuster to translate the beam in two dimensions.

6. The wireless power transmission system of claim 4 wherein the micro adjuster further comprises a second set of prism oriented to face each other, wherein disposed between the second set of prisms is a second distance that can be varied;
wherein the second distance determines the amount of translation of the beam through the second set of prisms; and
wherein the second set of prisms is oriented perpendicular to the first set to allow the micro adjuster to translate the beam in two dimensions.

7. The wireless power transmission system of claim 3 wherein the micro adjuster comprises a set of lenses for fine angular adjustment.

8. The wireless power transmission system of claim 1 wherein the transmitter further comprises:
a second beam partitioner and a beam detector that is connected to the control system, where the second beam partitioner partitions the reflected beam into at least two parts: a first part of the reflected beam that hits the position sensor and a second part of the reflected beam that hits the beam detector; and
wherein the beam detector sends a beam detector signal to the control system that contains characteristics of the reflected beam.

9. A wireless power transmission system of claim 8, wherein the beam modification signal is based on the beam detector signal.

10. A wireless power transmission system of claim 9, wherein the beam modification signal commands the beam source to modify the beam's intensity.

11. A wireless power transmission system of claim 9, wherein the beam modification signal commands the beam source to modify the beam's focus.

12. A wireless power transmission system of claim 1, wherein the reflector is selected from a group consisting of: a single corner cube retro-reflector, an array of single corner cube retro-reflectors, a partially transmitting mirror, a dichroic mirror, reflective paint, retro-reflective beads, glass, retro-reflective paints and combinations thereof.

13. A wireless power transmission system of claim 1, wherein the reflector has at least two regions that differ from each other in reflectance.

14. A wireless power transmission system of claim 13, wherein the at least two regions cause the reflected beam to have an identifiable intensity distribution and the beam adjustment signal is based on the identifiable intensity distribution.

15. A wireless power transmission system of claim 1, wherein the reflector is dynamically reflective.

16. The wireless power transmission system of claim 1, wherein the beam comprises at least two frequencies of electromagnetic energy and the beam partitioner separates the at least two frequencies.

17. The wireless power transmission system of claim 16, wherein one of the at least two frequencies is used for a function selected from a group consisting of: tracking, power absorption, data transmission, data reception, and combinations thereof.

18. The wireless power transmission system of claim 16, wherein one of the at least two frequencies is reflected by the reflector.

19. The wireless power transmission system of claim 1, wherein the reflector and the beam partitioner are a single structure, and the single structure is selected from a group consisting of: a single corner cube retro-reflector, an array of single corner cube retro-reflectors, a partially transmitting mirror, a dichroic mirror and combinations thereof.

20. The wireless power transmission system of claim 1, wherein the reflector and the beam partitioner are a single structure, and the single structure comprises two arrays of single corner cube retro-reflectors oriented with their back sides facing each other, wherein disposed between the two arrays is a distance that can be varied, and the distance determines the amount of reflectance of the single structure.

21. The wireless power transmission system of claim 19, wherein the single structures has at least two regions that differ from each other in reflectance.

22. The wireless power transmission system of claim 21, wherein the at least two regions cause the reflected beam to have an identifiable intensity distribution and the beam adjustment signal is based on the identifiable intensity distribution.

23. The wireless power transmission system of claim 1, further comprising a power consumption device connected to the energy collector.

24. The wireless power transmission system of claim 1, further comprising a charging system connected to the energy collector.

25. The wireless power transmission system of claim 1, wherein the energy collector is partially transmissive.

26. The wireless power transmission system of claim 1, wherein the transmitter further comprises a modulator to encode data on the beam.

27. The wireless power transmission system of claim 1, wherein the receiver further comprises a modulator to encode data on the reflected beam.

28. The wireless power transmission system of claim 27 wherein the modulator is selected from a group consisting of: a liquid crystal display (“LCD”), a non-LCD optical modulator, a dynamic layer, a parallel plate modulator, and combinations thereof.

29. The wireless power transmission system of claim 27 wherein the modulator comprises two arrays of single corner cube retro-reflectors oriented with their back sides facing each other, wherein disposed between the two arrays is a distance that can be varied, and the distance determines the amount of reflectance of the modulator.

30. The wireless power transmission system of claim 1, wherein the transmitter further comprises a de-modulator to decode data from the reflected beam.

31. The wireless power transmission system of claim 1, wherein the receiver further comprises a de-modulator to decode data from the beam.

32. The wireless power transmission system of claim 1, wherein the receiver further comprises a beam spreader to increase the angle of acceptance of the receiver.

33. The wireless power transmission system of claim 1, wherein the receiver further comprises a beam spreader to increase energy absorption of the receiver.

34. The wireless transmission system of claim 1 wherein the beam is selected from a group consisting of: thermal, laser, gas discharge, arc, and combinations thereof.

35. The wireless transmission system of claim 1 wherein the beam source further comprises at least one beam modifying component selected from a group consisting of: a beam generator, beam concentrator, beam collimating optics, a beam intensity control, beam conditioning optics, and combinations thereof.

36. A method for wireless power transmission in a system comprising a transmitter and a receiver, comprising the steps of:
a. transmitting a low-powered and defocused beam from the transmitter;
b. reflecting at least a portion of the beam from the receiver back to the transmitter;
c. monitoring for the detection of a reflected beam at the transmitter;
d. once the presence of a reflected beam is detected, focusing the beam and directing the beam to the area where the reflected beam was detected;
e. adjusting the beam with macro and micro adjusters until a reflected beam with desired characteristics is detected at the transmitter;
f. once the reflected beam with desired characteristics is detected at the transmitter, increasing the power of the beam;
g. adjusting the beam with macro and micro adjusters to maintain the desired characteristics of the reflected beam, as detected at the transmitter; and
h. powering down the beam when the characteristics of the reflected beam, as detected at the transmitter, become undesirable.

37. The method of claim 36 wherein steps (e) and (g) further comprise modifying the beam with at least one beam modifying component comprises at least one beam modifying component selected from a group consisting of: a beam generator, a beam concentrator, beam collimating optics, a beam intensity control, beam conditioning optics, and combinations thereof.

38. The method of claim 36 wherein step (h) the undesirable characteristic is the absence of a detected reflected beam at the transmitter.

39. The method of claim 36 wherein the macro adjusters are angular and the micro-adjusters are translational.

40. The method of claim 39 wherein the translational adjuster comprises:
a first set of prisms oriented to face each other, wherein disposed between the first set of prisms is a distance that can be varied; and
wherein the distance determines the amount of translation of the beam through the first set of prisms.

41. The method of claim 36 wherein the micro adjuster comprises a set of lenses for fine angular adjustment.

42. The method of claim 36 wherein the desirable characteristic is determined using an intensity distribution measurement of the reflected beam.

43. The method of claim 42 wherein the receiver comprises a reflector with at least two regions of reflectance and the intensity distribution corresponds to the at least two regions of reflectance.

44. The method of claim 36 wherein the receiver comprises a reflector that has a dynamically assignable reflectance.

45. The method of claim 36 wherein the receiver is passive and receives substantially all its power from the beam.

46. The method of claim 36 wherein step (h) is performed by a position sensor and a control system.

47. The method of claim 36 wherein the transmitter comprises a position sensor connected to a control system and wherein step (h) is performed by a position sensor, beam detector and a control system.

48. The method of claim 36 wherein the transmitter comprises a beam detector connected to a control system and a position sensor connected to the control system and wherein method further comprises after step (h), directing the low powered beam to the region where the reflected beam was last detected and repeating steps (e) through (h).

49. The method of claim 36, wherein step (g) further comprises using predictive algorithms.

50. The method of claim 36 wherein step (f) further comprises modulating the beam between a high power transmission period and a low power transmission period, and wherein steps (e) through (h) are performed during the low power period.

51. The method of claim 36 wherein the transmitter modulates the beam and the transmitter authenticates the reflected beam by detecting the modulated beam.

52. The method of claim 36 wherein the receiver modulates the reflected beam to encode data and the transmitter de-modulates the beam to decode the data.

53. The method of claim 52 wherein the transmitter authenticates the receiver based on the decoded data.

54. The method of claim 36 wherein the transmitter modulates the beam to encode data and receiver de-modulates the beam to decode the data.
